(19) United States
(12) Patent Application Publication
(10) Pub. No.: US 2024/0353320 A1
(43) Pub. Date: Oct. 24, 2024

(54) OPTICAL SYSTEM AND METHODS OF USE

(71) Applicant: Boehringer Ingelheim Vetmedica GmbH, Ingelheim am Rhein (DE)

(72) Inventors: Axel NIEMEYER, Ingelheim am Rhein (DE); Matthias GRIESSNER, Ingelheim am Rhein (DE); Ricardo OSÓRIO, Porto (PT)

(21) Appl. No.: 18/575,214

(22) PCT Filed: Jun. 24, 2022
(86) PCT No.: PCT/EP2022/067374

§ 371 Date: Dec. 28, 2023

(30) Foreign Application Priority Data
Jul. 1, 2021 (EP) 21183295.1

(51) Int. Cl. G01N 21/25; G01J 3/02; G01J 3/44; G01N 21/31; G01N 21/33; G01N 21/359; G01N 33/487

(52) U.S. Cl. CPC G01N 21/255; G01J 3/0218; G01J 3/4406; G01J 3/4412; G01N 21/33; G01N 21/359; G01N 33/487; G01N 2021/3155; G01N 2201/0624; G01N 2201/0627; G01N 2201/08

(57) ABSTRACT

An optical system having a chamber for receiving an element of body fluid or tissue or environmental sample to be characterized has a light source for illuminating the chamber with light, and a spectrometer for recording a spectrum of light originating from the chamber. The light source has two separate LEDs to emit light having at least two spectral maxima of different wavelength ranges. The light from the light source is directed to the chamber. A method for determining a parameter representing a property of the element with the optical system, wherein, light having at least two spectral maxima of different wavelength ranges generated by separate LEDs is directed onto the element, a spectrum with reflected components of the light, scattered components of the light, and/or light caused by Raman scattering or fluorescence of the element is measured with the spectrometer, and the parameter is determined by evaluating the spectrum.

### BACKGROUND OF THE INVENTION

### Field of the Invention

The present invention relates to the field of biophotonic equipment applied to healthcare; in particular, it relates to a biophotonic device for the point-of-care, real-time, non-invasive determination of parameters with diagnostic relevance, in particular of tissue (e.g., blood, adipose tissue) samples, in-vivo (e.g., by skin contact), environmental samples preferably comprising one or more of bacteria, viruses, protozoa, parasites, fungi, plants, substances, in particular metabolic products, derived therefrom like mycotoxin, or the like.

### Description of Related Art

Current biophotonic systems present a series of technical limitations as regards use as a real-time holistic health system at the point-of-care. It is one goal of the present invention to improve such systems.

International Patent Application WO 2014/118745 A1, which is incorporated herein by reference in its entirety, relates to an optical system for parameter characterization of an element of body fluid or tissue comprising a spectrometer for recording the spectrum of light from the element and an optical device which comprises a light source for emitting light onto the element. The optical system according to the present invention can be, can be similar or can have some or all features of the optical system as disclosed in WO 2014/118745 A1, i.e., can be an improved version thereof, but can also realized and favorable in different contexts.

### SUMMARY OF THE INVENTION

The object of the present invention is to provide a method and an optical system with improved performance.

This object is achieved by an optical system as described herein.

One aspect of the present invention relates to an optical system having a chamber for receiving an element of body fluid or tissue or environmental sample to be characterized by the optical system, a light source for illuminating the chamber with light, and a spectrometer for recording a spectrum of light originating from the chamber.

According to this aspect, the light source comprises at least or exactly two separate LEDs to emit light having at least two spectral maxima of different wavelengths/in different wavelength ranges, and the light source is coupled to the chamber such that the light is guided and/or directed from the light source to the chamber when the light source is activated.

One aspect of the present invention, which can be combined with the previous aspect or realized independently as well, relates to a method for determining a parameter—representing a property of an element of body fluid or tissue or environmental sample—with an optical system, wherein the optical system comprises a chamber for receiving an element of body fluid or tissue or environmental sample to be characterized by the optical system, a light source for illuminating the chamber with light, and a spectrometer for measuring a spectrum of light originating from the chamber.

In order to determine the parameter, light having at least two spectral maxima of different wavelengths/in different wavelength ranges are generated by separate LEDs and is directed onto the element, a spectrum comprising reflected components of the light, scattered components of the light, and/or light caused by Raman scattering or fluorescence of the element is measured with the spectrometer, and the parameter being determined by evaluating the spectrum.

It has been turned out that surprisingly use of at least or exactly two LEDs is particularly efficient for exact correlation when examining an element of body fluid or tissue or environmental sample. In particular, use of at least or exactly two LED enables analysis of particular substances that were not able to be identified by LED based techniques before.

A chamber according to the present invention preferably is a holder for receiving the element, in particular a sample of body fluid or tissue or environmental sample, to be characterized by the optical system comprising a light source for emitting light onto the element.

The chamber can be comprised in a disposable capsule. The chamber can comprise a chemical or biological marker or not. The chamber preferably comprises one or more mirrors for reflecting the light from the light source through the element to be characterized to the spectrometer, in particular the mirror or mirrors being attached to the chamber lid or lids. One mirror in the chamber can be arranged distal an optical device-comprising at least the light source and the spectrometer—to reflect light back to the optical device, and comprising one mirror coupled to the optical device said mirror being arranged to reflect light back to the element to be characterized.

The chamber can be attachable to the optical device for characterization of the element, preferably by magnets or by mechanical pressure or mechanical fasteners. The chamber preferably is liquid-tight, in particular by enclosing with one or more lids, said enclosure being liquid-tight, which can be disposable. However, different solutions can be possible.

An element according to the present invention preferably is a body in-vivo element, in particular wherein the optical system is arranged for parameter characterization of the body element by means of the optical device. It can be a sample of body fluid, in particular blood, blood serum, saliva, sweat, urine or tears, or a sample of body tissue, in particular adipose tissue.

A body fluid or tissue according to the present invention preferably is a substance or sample taken from the body of a human or animal, preferably covering process fluids comprising an in particular processed, more specifically crushed, substance or sample taken from the body of a human or animal.

Alternatively, or additionally, the element can be or comprise an environmental sample. Such environmental samples preferably comprise one or more bacteria, viruses, protozoa, parasites, fungi, plants, or substances, in particular metabolic products, derived or produced therefrom like mycotoxin. Environmental samples preferably can be obtained by extraction, filtration of a substance that can be taken from the environment. Environmental samples in particular are feed samples.

A light source according to the present invention preferably is configured to emit light to, in particular into, the chamber. The light source preferably is or comprises one, two or multiple LEDs. However, the light source can be or comprise alternatively or additionally a bulb, a laser diode or different device for generating electromagnetic radiation in the light range including IR and UV, e.g., by transmittance, reflectance, scattering, Raman scattering or fluorescence of the emitted light by said element and/or the chamber.

The light source alternatively or additionally can comprise one or more light emitters and/or one or more light filters in order to emit different spectra or a spectrum comprising at least two maxima, one in the UV range and one in the IR and/or VIS range (each).

In one embodiment, the light source can be configured to select or change the wavelength maximum or maxima of emitted light. For example, this can be achieved by selective activation of only particular of multiple light sources and/or filtering.

Illuminating according to the present invention preferably means emission of light into the chamber, such that the light can fall onto the element if received in the chamber, and/or parts of the chamber inner surfaces, in particular including at least one mirror. Said emitted light at least partially can be provided to the spectrometer.

The light source preferably comprises at least one LED-Light Emitting Diode. An LED is semiconductor light source that emits light when current flows through it. Typically, an LED produces exactly one power maximum. However, LEDs alternatively or additionally might produce one main maximum having the highest power and a secondary maximum with less power than the main maximum. In particular, LEDs for producing a maximum in the range visible to the human eye typically have one maximum at the color they produce. UV LEDs, however, might produce the light in the UV wavelength range by multiplication of (N) IR light. In such cases, a secondary maximum might in the (N) IR wavelength range. Use of such LEDs is preferred in the present invention as it has been turned out surprisingly that this has a positive synergistic influence on the overall performance.

In the sense of the present invention, the term “LED” covers semiconductor lasers, laser LEDs or different light sources that emit light based on the principles LEDs use for emitting light.

A (spectral) maximum, also referred to as emission maximum, preferably is a maximum of spectral power (in mW) of light. In the sense of the present invention, a maximum typically is a peak that might have shoulders or the like, but essentially is a spectral line that can be bell-shaped and/or narrow band. A spectral maximum preferably has a line width (ΔE or FWHM—full width half maximum) of less than 10 kT, preferably less than 6 kT, in particular approximately 1.8 kT; and/or Δλ of less than 10 kT*λ2/(hc), preferably less than 6 kT*λ2/(hc) in particular approximately 1.8 kT*λ2/(hc). Thus, the line-width of an LED emitting in the visible range, the UV range or the (N) IR range in the sense of the present invention is relatively narrow compared with the range of the entire visible spectrum, the UV spectrum, or the (N) IR spectrum, respectively.

The spectrum preferably can be contained within UV-VIS-NIR wavelengths, in particular wavelengths of 200-2500 nm, further in particular wavelengths of 200-400 nm. In an embodiment, the spectrometer comprises a CCD sensor.

In particular, UV wavelength range is from 100 nm to 380 nm wavelength, and/or VIS wavelength range is from 380 nm to 780 nm, and/or IR wavelength range is from 780 nm to 1 mm, and/or NIR wavelength range is from 780 nm to 1400 nm.

A wavelength range in the sense of the present invention preferably also is referred to as spectral range or frequency range of electromagnetic waves/light.

A brightness according to the present invention preferably is or represents the amount of light provided by the light source. In particular, the brightness is or corresponds to the luminous intensity (a photometric measure of luminous flux per unit solid angle) or luminous flux (a photometric measure of luminous energy per unit time) or luminance (a photometric measure of the luminous intensity per unit area) of light emitted from the light source or light radiated into the chamber. From the brightness, the amount of light that passes through, is scattered by, or is reflected from the chamber and/or the element contained in the chamber depends. Any change in brightness of the light source preferably results in an, in particular approximately proportional, change of the amount of light falling onto the spectrometer, i.e., the sensor measuring the spectrum.

Illuminating according to the present invention preferably means emission of light into the chamber, such that the light can fall onto the element if received in the chamber, and/or parts of the chamber inner surfaces, in particular including at least one mirror. Said emitted light at least partially can be provided to the spectrometer.

A spectrometer according to the present invention preferably is or comprises a sensor for measuring a spectrum of light received by the spectrometer, i.e., falling onto the sensor. The spectrometer outputs or provides for retrieval data representing the spectrum. According to the present invention, a spectrum and data representing said spectrum comply to each other and, thus are used interchangeable. The spectrum has values for multiple wavelengths or corresponding frequencies.

The spectrometer preferably is configured for recording the spectrum of light from the chamber, said light from the chamber being of transmittance, reflectance, scattering, Raman scattering or fluorescence of the emitted light by said element and/or the chamber.

Fiber optics according to the present invention preferably is a means for guiding light. In particular, it is a glass fiber or optical fiber from a different, light transmitting material preferably having the shape of a cable (optical fiber cable). Thus, multiple fiber optics are in particular multiple fiber cable for guiding light, respectively.

The spectrum preferably can be contained within UV-VIS-NIR wavelengths, in particular 200-2500 nm wavelengths, further in particular 200-400 nm wavelengths. In an embodiment, the spectrometer comprises a CCD sensor.

A control or to control the light source according to the present invention preferably is either a feed forward or feedback control or a combination of feed forward and feedback control of the light source regarding its brightness, i.e., preferably regarding its luminous intensity of light radiated into the chamber or a luminance of the light source.

To control of the light source in the sense of the present invention, accordingly, preferably means to feed forward or feedback control or a combination of feed forward and feedback control of the light source for maintaining or changing a particular brightness of the light source. In particular, control of the light source means feed forward or feedback control or a combination of feed forward and feedback control in order to reduce the brightness about a particular extend or to obtain a particular brightness.

Feedback control preferably means in the sense of the present invention that a power, a luminous intensity or luminance of or related to the light source/LED or the light originated from the chamber is compared to a target/preset like the maximum output of the spectrometer or the dynamic range maximum of the spectrometer. For that purpose, it can be measured by the spectrometer or a different sensor. The light source/LED is controlled based on the outcome of that comparison, preferably such that a difference between the measured value and the target/preset is reduced or eliminated. Because controlling the light source/LED brightness influences the measured value and again the brightness control, this is referred to as control loop.

Free of overdrive is a sensor and, in particular the spectrometer, if the dynamic range is not exceeded. When exceeding the dynamic range, the sensor outputs a maximum value regardless of whether this value is exceeded or not. Further, the sensor might be affected. Thus, exceeding the dynamic range preferably is avoided. However, using only a small portion of the whole dynamic range can cause lost of resolution. Thus, using essentially the entire dynamic range is preferred. This was unusual in the field of the present invention in particular during calibration.

The invention can comprise method steps or the optical system of the present invention can comprise a data processing module configured to carry out steps to:
convert the recorded spectrum by a correction variable, in particular a conversion matrix, into a standardized spectrum, wherein said correction variable or conversion matrix has been obtained by calibrating (comparing) the optical system spectrum response (with empty chamber) against a spectrum reference;
to pre-process the converted spectrum; and
to correlate, for parameter quantification, the converted pre-processed spectrum with pre-obtained spectral bands for each parameter.

Calibration according to the present invention preferably is a method for compensating at least to some extend errors or inaccuracies. The calibration can be carried out against a spectrum reference previously measured by a reference spectrometer. The pre-obtained spectral bands for each parameter can be previously measured by the reference spectrometer. The reference spectrometer has improved or equal optical resolution, noise rejection, or light sensitivity in comparison with the optical device spectrometer.

It is preferred to use the original light source, i.e., the light source used for later measurements, as well as for calibration (through the mirror). Hence, it can be avoided to make use of different hardware references (blanks) across the same or different entities of the optical system.

For example, multiple copies of different exposures of one particular and/or various samples can be compared to one another. Based thereon, the quality of the exposures can be determined. Depending on said quality, i.e., when a minimum threshold or an optimum dynamic range of the exposure is reached, preferably without exceeding the dynamic range, a (recommendation for) calibration is determined or triggered based thereon.

The darker the sample is, the higher the intensity of the light provided by the light source preferably is chosen, particularly such that at least essentially the same exposure time and/or light intensity (for the sensor) is achieved. The geometry preferably is such, in particular an interface to the optical device like a glass and the mirror preferably are distal from each other in such a way, that the exposition time (for the sensor) is set to a minimum by intensity control of the light source.

Reducing the exposition time preferably reduces the noise-level of a sensor signal and/or a time to obtain results.

The sensor preferably measures the count of photons, further preferably in each particular wavelength (ranges). When the brightness is reduced, said count number is reduced, preferably to a maximum count the sensor supports, but not exceeding it. That is, as already said, the brightness is reduced to an intensity that complies with the dynamic range of the sensor.

In particular the exposition time the sample/sensor is set to a minimum supported by the sensor while the light intensity at least essentially is fitted to the dynamic range of the sensor.

Further aspects of the present invention can be gathered from the claims and the following description of preferred embodiments. The following figures provide preferred embodiments for illustrating the description and should not be seen as limiting the scope of the disclosure.

### BRIEF DESCRIPTION OF THE DRAWINGS

FIGS. 1a and 1b show a perspective view of the handheld and benchtop photonic system, with all the components, how they are assembled and connected to optionally perform the present disclosure;

FIG. 2 shows an optional plug-in/plug-out system for a chamber and corresponding attachment probes in which FIG. 2a shows a transmittance probe: FIG. 2b presents a micro-needle probe; and FIG. 2c shows a transmittance probe.

FIGS. 3a and 3b show the internal optic system to enable the plug-in/plug out system using UV-VIS-NIR bulbs or led diodes, and laser diodes, respectively; and FIG. 3c shows basic components of the optical system:

FIG. 4 is a schematic drawing of embodiment for a probe, in which a capsule comprises a chamber for receiving a sample:

FIG. 5 is a simplified schematic of the optical system;

FIG. 6 shows a spectrum with varying brightness of the light source:

FIG. 7 is a simplified schematic of spectra of two LEDs, and of a measured spectrum (without reduced brightness);

FIG. 8 is a simplified schematic of spectra of two LEDs, and of a measured spectrum (with reduced brightness):

FIG. 9 is a flow chart of a calibration procedure:

FIG. 10 is a flow chart of a measurement procedure:

FIG. 11 shows graphs of a measured and a reference spectrum as well as a graph showing their difference; and

FIG. 12 shows graphs of a measured and a reference spectrum showing the spectral maxima.

### DETAILED DESCRIPTION OF THE INVENTION

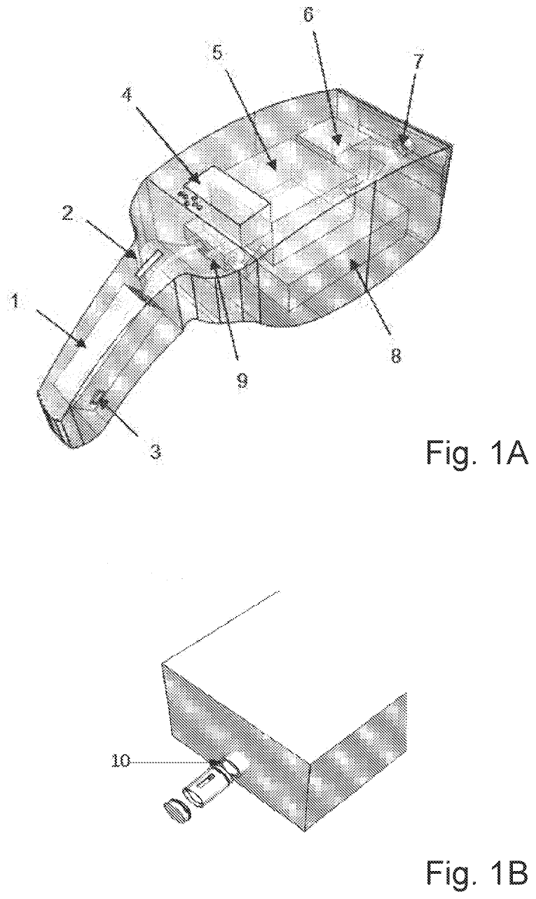

An embodiment of a modular point-of-care photonic system is presented in FIG. 1. This figure presents the handheld system and, in detail, a plug-in/plug out magnetic system, where sterile probes can be directly attached. The present invention can be advantageously realized with such system, but does not need to and, in particular, several features do not need to be realized.

The system is comprised of: miniaturized personal computer 1 (ram memory, flash disk, wireless communications, USB connection, CPU): micro USB hub 2: USB 3 (recharge and connection): LCD display and control 4: Light source 5 (led, light bulb or laser diode); spectrometer 6: magnetic plug-in/plug-out system 7: Optical bench 8 (connecting fiber optics); Lithium ion battery 9; and fast magnetic or pressure attachment for modular probes 10 (reflectance, mini-needle and transmittance).

The fast magnetic attachment system possesses the correct polarity in order to attract the probes or pressure tips to ensure perfect plugging as possible. Any type of mechanical fastener or mechanical coupling may also be used, mechanisms such as rotate-to-lock, clip-to-lock, slide-to-lock, among others.

All probes and the attachment system are preferably made of surgical grade steel or alternatively plastic for disposable kits.

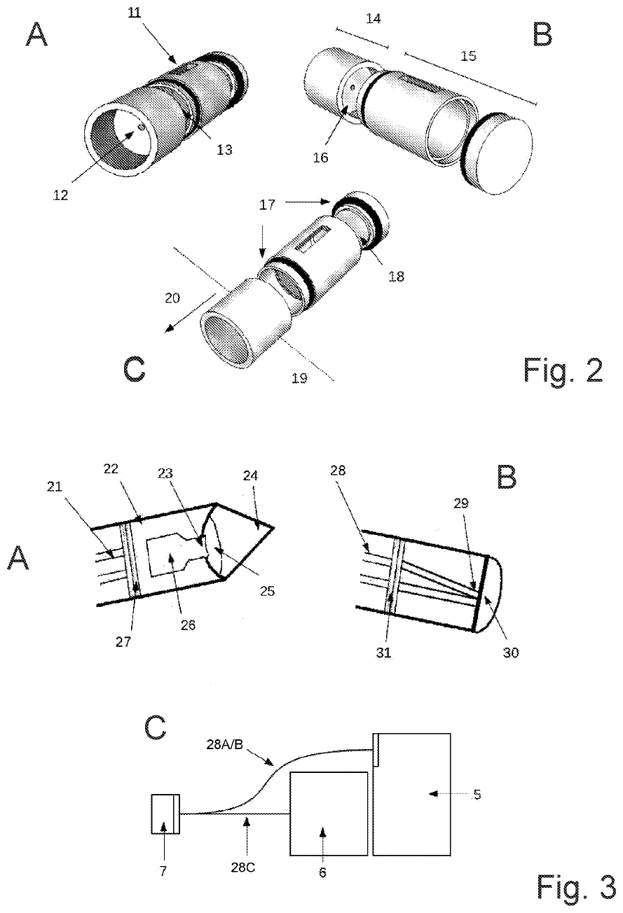

FIG. 2 shows an embodiment of a transmittance probe. This probe is designed so that the light enters a window 12 and passes through a glass/capsule wall 13 and through the element E/samples. The optional misalignment of the input and output fiber in the window 12, 16 is purposeful such that light will be forced to be reflected inside the chamber 26 a large number of times, so that the light path is significantly increased, and as a consequence, also signal sensitivity. Furthermore, as absorbed light is re-emitted in all directions, a large proportion of it will escape through the top exit hole, and not enter the reception fiber slit, greatly increasing the difference between the emitted light and sample spectrum fed back to the receiver, and thus, sensitivity.

The element E, e.g., a liquid sample can be put inside the chamber 26 in particular by the hole 11. The fast attachment occurs preferably due to the magnets, o-rings or pressure plug 17, attaching sections 14 and 15. The mirror 18 may also be detachable from the main part of the chamber 26 for, e.g., better sterilization and avoidance of cell or calcium deposits. Furthermore, the axis of the attachment system 19, and the direction 20 are depicted. The chamber volume is usually less than 1 ml.

The attachment of the probe with the chamber 26 to the optical device can be alternatively carried out by mechanical pressure or mechanical fasteners, as mentioned above.

FIG. 3a presents an embodiment with micro-needle and an embodiment of diffusive reflectance probes, as well as, the optical bench. The micro-needle probe is preferably composed of one or more of: optical bench 21 that can comprise or be formed by fiber optics 28: steel capsule or other suitable material 22, in particular plastic for disposability: micro-channel 23; puncturing tip 24: mirror(s) 25, in particular one mirror, distal from the main system: (internal) chamber 26 (and opening) and fast plug-in/plug out system 27, in particular equal to any of the previously described for the transmittance probe.

After puncturing the skin, a small drop of blood is channeled into the measuring chamber 26, where the measurement is taken. In particular, the needle or puncturing part 24 of the probe is connected to the (internal) chamber 26 through an opening in the distal mirror 25.

An embodiment of the transmittance probe of FIG. 3b comprises of fiber optics 28: illuminating and center capture fiber optics 29: focusing lenses 30; and fast plug-in/plug out system 31, preferably equal to any of the previously described for the transmittance probe. This probe 3b measures the light reflected from the specific focus point inside a tissue or other element E that optionally can be taken into a chamber 26 (not shown). Different lenses can provide different focusing reflectance distances for spectral measurements at pre-determined depths of samples or body parts with no invasion of the body by pointing (e.g., from a distance, usually small distances, limited by the sensitivity of the system), in particular touching, at the surface (skin, in most situations, but other surfaces are possible, namely different mucosa). Focusing mechanisms provide the ability to perform automatic scanning characterization at different distances.

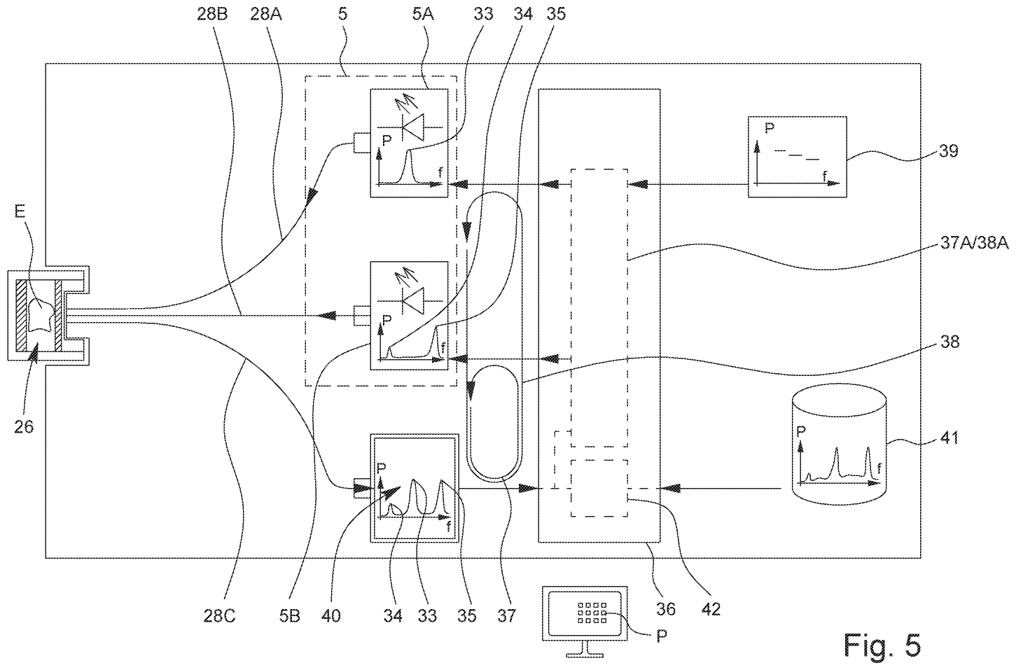

FIG. 3c shows the optical system's 1 basic components light source 5, optional plug-in/plug-out system for coupling the capsule 22/chamber 26 which however can be coupled differently in the alternative, spectrometer 6 and fiber optics 28 connecting them optically further discussed in detail with regard to FIG. 5.

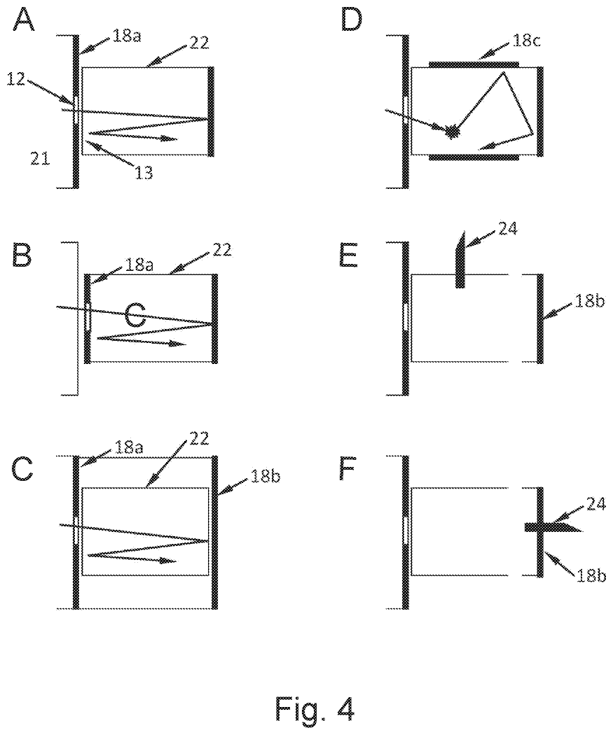

An aspect of the disclosure is the probe used with the optical system 1. FIG. 4 shows additional embodiments for the probe, in which a capsule 22 comprises a chamber 26 for receiving the element E (sample).

In an embodiment of FIG. 4a, the optical bench 21 (emitter-fiber optics 28 towards the spectrometer 6—and receiver—fiber optics from the light source 5) transmits/receives through a window 12 in a mirror 18a rigidly coupled to the optical bench 21 part of the system. Capsule 22 comprises a transparent window/capsule wall/glass 13 in the part proximal to the optical bench 21, that allows the emitted light to pass through to the chamber 26 and to return back to the receiver. A second mirror 18b is provided in the capsule 22 distal to the optical workbench 21. This way, the emitted light is able to reflect multiple times between the mirrors 18 before being reflected back to the receiver and thus amplify the signal received. Preferably, the emitter, receiver and/or mirrors 18 are aligned such that light will travel between emitter and receiver such that the number reflections are maximized. The second mirror 18b being in the capsule 22 has the advantage that light will not pass through the capsule wall when being reflected by this second mirror 18b, thus improving the signal quality.

In an embodiment of FIG. 4b, the first mirror 18a, closest the workbench 21, is alternatively located in the capsule 22 in the part proximal to the optical bench 21. This has the advantage that, compared to the previous embodiment, reflected light at the first mirror 18a does not pass multiple times through a capsule wall/window 13 improving signal quality. If light passes multiple times through a capsule material the signal may be distorted by the capsule material this may not be possible to be compensated by software). It has the disadvantage that construction is not as simple as for the previous embodiment and the cost of the capsule is higher, an issue if the capsule 22 is disposable (not reusable).

In an embodiment of FIG. 4c, both mirrors proximal 18a, distal 18b are provided coupled to the optical workbench 21, and the capsule 22 does not include any mirrors but simply transparent walls next to said mirrors 18a, 18b such that light might be reflected from emitter to receiver multiple times through the sample. It has the advantage that construction is simpler, as compared to the previous embodiments, and the cost of the capsule 22 is lower, an important advantage if the capsule 22 is disposable (not reusable. It has the disadvantage that light passes multiple times through a capsule material (twice as much as embodiment ‘a’) and the signal may be distorted by the capsule material (even if this may be possible to partially compensate by software in some cases).

In an embodiment of FIG. 4d, the capsule 22 may be provided with lateral mirrors 18c relative to the reflection of the light path. This is usually an advantage in Raman modes of operation where re-emitted light (in all possible directions) is of interest.

For transmittance modes of operation, lateral mirrors are usually disadvantageous as re-emitted light is of no interest in this case—the absence or reduction of lateral reflection is of interest in that the embodiment promotes reflection of transmittance light (direction perpendicular to the lateral surfaces) over reflection of re-emitted light (all directions).

In an embodiment of FIG. 4e the capsule 22 is provided with a puncturing tip 24 for obtaining the element E/fluid (for example, blood) sample into the chamber 26. Alternatively, instead of a puncturing tip 24, an opening may be provided, optionally closable with a lid. Alternatively, a part of the capsule 22 wall may be puncturable, for example by a syringe, for receiving the element E (fluid sample). Alternatively, the capsule 22 wall comprising the distal mirror 18b may be detachable for receiving the element E (fluid sample). These options may be freely combined for providing multiple options to the user.

In an embodiment of FIG. 4f, the puncturing tip 24 is located at the capsule 22 wall comprising the distal mirror 18b, said wall being detachable for receiving the element E (fluid sample). This way the part of the capsule 22 comprising the puncturing tip 24 may be disposable, while the rest can be re-used. Alternatively, in another embodiment E, the puncturing tip 24 is located in a capsule 22 wall that does not comprise the distal mirror 18b, said wall of the distal mirror 18b being also detachable for receiving the element E (fluid sample). This way the disposable part does not include a mirror and the cost for re-use may be lower.

The chamber 26 of the capsule 22 may be pre-provided with chemical or biological markers, for example genetic markers, such that the fluid sample mixes with said marker(s). A marker generally refers to a measured characteristic which may be used as an indicator of some chemical or biological parameter. This way, specific parameters which cannot be obtained through the spectra received from the sample, can now be detected as long as said markers make apparent in the recorded spectra said parameters. For example, it is advantageous to provide specific coloring markers able to provide a significant spectrum change on the presence of elements which would normally be transparent to the light frequencies herein used.

The internal optical bench 8 is, in an embodiment, composed of the fast plug-in/out system 32 with to each at least two internal fiber optics 28 are linked: the fiber optics 28A, 28B conduct the light from the light source 5; and the fiber optics 28C conducts light into the spectrometer 6.

FIG. 5 shows an optical system S comprising the chamber 26 for receiving an element E of body fluid or tissue or environmental sample to be characterized by the optical system S. Further, the optical system S comprises at least one light source 5 for illuminating the chamber 26 with light. Moreover, the optical system S comprises the spectrometer 6 for recording a spectrum of light originating from the chamber 26.

For measuring the spectrum 40, as shown in FIGS. 8 and 12, the light source 5 preferably emits a spectral maximum in the ultra violet (UV) range 35 and at least one further spectral maximum in the spectral range visible to the human eye (VIS) 33 and/or at least one further spectral maximum in the (near) infrared (IR) range 34.

The light source 5, thus, is configured to emit light having at least two spectral maxima 33, 34, 35 of different wavelength ranges UV, VIS, NIR. The light source 5 is coupled to the chamber 26 such that the light is directed from the light source 5 to the chamber 26 when the light source 5 is activated.

The optical system S preferably comprises at least three fiber optics 28, wherein light with the spectral maxima 33, 34, 35 of at least two different wavelength ranges UV, VIS, NIR is guided to the chamber 26 via a first fiber optics 28A and a second fiber optics 28B while light is guided from the chamber 26 to the spectrometer 6 via a third fiber optics 28C of the three-fiber optics 28.

The light source 5 is configured to generate light with spectral maxima 33, 34, 35 of a wavelength, preferably, in a spectral maximum in the UV range 35 (ultraviolet spectral range) and in the VIS range 33 (spectral range visible to the human eye).

Particularly preferably, each of the spectral maximum in the UV range 35 and the spectral maximum in the VIS range 33 are emitted by means of (different) LEDs 5A, 5B which in the embodiment of FIG. 5 are depicted schematically by means of a respective symbol and a schematic, exemplary diagram of power P over frequency f to indicate the emitting spectrum.

The light source 5, in particular by means of the LED 5B, can be configured for generating and/or emitting light with the spectral maximum in the UV range 35 in addition to generating light with a spectral maximum in the IR range 33 (infrared spectral range), in particular NIR range (near-infrared).

The UV LED 5B is preferably coupled to the chamber 26 via fiber optics 28B via which the light generated or producible by the UV LED 5B with spectral maximum in the UV range 35 and in addition a spectral maximum in the IR range 33 is guided to the chamber 26 while being superimposed.

In particular, UV spectral range is from 100 nm to 380 nm wavelength, VIS spectral range is from 380 nm to 780 nm wavelength, IR spectral range is from 780 nm to 1 mm wavelength, wherein NIR spectral range is from 780 nm to 1400 nm wavelength.

Surprisingly it has been turned out that, in particular for analyzing elements E of body fluid or tissue or environmental sample that it is favorable for a compact and energy saving construction to make use of LEDs while making use of at least two LEDs with a spectral maximum in the UV range 35 on the one hand and in the VIS range 33 on the other hand results in particular meaningful measurable spectra 40 for calibration and/or measurement.

The spectrometer 6 preferably comprises a brightness control 36 for controlling the brightness of the light source 5.

The brightness control 36 preferably is designed to control the brightness, in particular to control it by means of the light source 5 and the spectrometer 6, in such a way that the spectrometer 6 is operated at least substantially free of overdrive and/or up to the limit of its dynamic range preferably at at least one of the wavelength ranges UV, VIS, NIR, preferably at least two of the wavelength ranges UV, VIS, NIR, or their maxima 33, 34, 35.

In particular, the brightness control 36 is designed to individually control the brightness of at least two different of the wavelength ranges UV, VIS, NIR or of its maxima 33, 34, 35 in such a way that the spectrometer 6 at at least one of the wavelength ranges UV, VIS, NIR, preferably at at least two wavelength ranges UV, VIS, NIR is driven at least substantially free of overdrive and/or up to the limit of its dynamic range.

The brightness control 36 for controlling the brightness of the light source 5, in particular one or more of the LEDs 5A, 5B, preferably forms at least one control loop 37, 38. The brightness control 36 can be coupled to the spectrometer 6 for detecting the brightness of the light measured with the spectrometer 6, and the brightness control 36 is coupled to the light source 5 for controlling it in such a way that the brightness of the light does not overdrive the spectrometer 6, preferably while driving it substantially to its dynamic range limit.

In particular, the brightness control 36 controls the light source 5 on the basis of a comparison of the brightness measured with the spectrometer 6 (or a different brightness sensor) with a reference variable 39 representing the dynamic range of the spectrometer 6, preferably so that the spectrometer 6 at least substantially is driven free of overload and at at least one of the wavelength ranges UV, VIS, NIR and/or at least substantially up to the limit of its dynamic range.

The brightness of the light with the wavelength in the UV and/or VIS range of wavelengths, i.e., the power of the spectral maximum in the UV range 35 and the power of the spectral maximum in the VIS range 33 preferably are separately controllable.

In particular, the brightness control 36 comprises at least two feedback control loops 37, 38 so that the spectrometer 6 is driven at at least two or all of the wavelength ranges UV, VIS, NIR or maxima 33, 34, 35 substantially free of overshoot and/or up to the limit of its dynamic range.

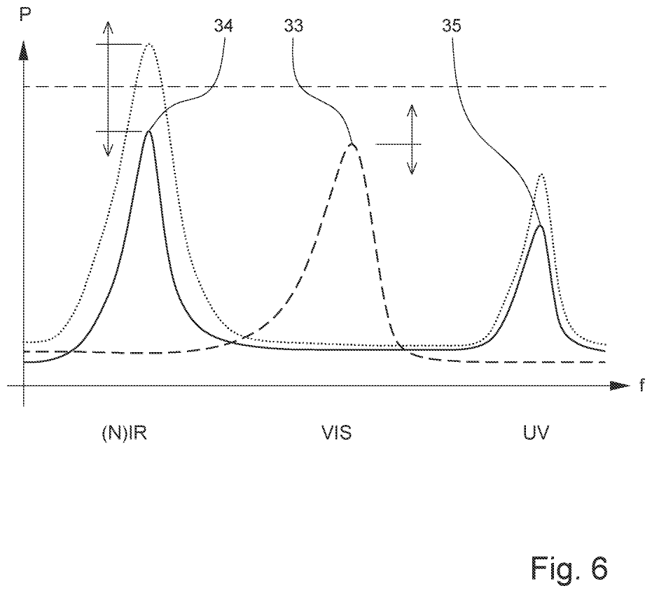

Referring to FIGS. 6 to 8, the brightness of the light source 5 preferably is controlled while measuring a spectrum 40 for calibration of the spectrometer 6 so that the brightness of the light source 5 is a reduced and/or (feedback) controlled brightness, a brightness reduced relative to a nominal brightness of the light source 5, and/or a brightness reduced relative to the brightness of the light source 5 when measuring the spectrum 40 of the element E.

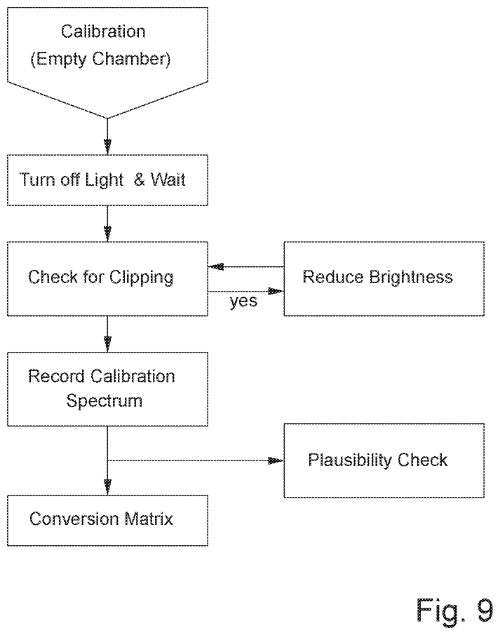

Referring to FIG. 9, a calibration preferably is performed with an empty chamber 26. The empty chamber 26 can be illuminated with the light source 5 while the spectrum 40 is measured. The reflection behavior of the empty chamber 26 differs significantly from the chamber 26 containing the element E such that brightness reduction has a particular advantageous effect in case of calibration with empty chamber.

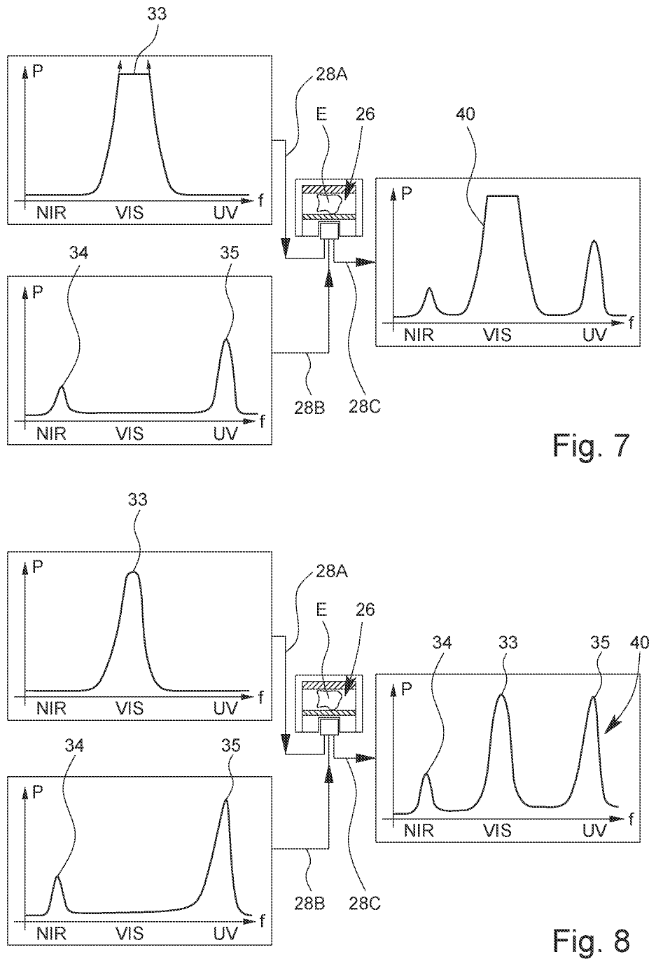

In a first step of calibration, the light source 5 is activated. In particular, both LEDs 5A, 5B are activated, in FIG. 7 represented by spectra having a maximum in the VIS range 33 on the one hand and spectrum having at least a maximum in the UV range 35 and, preferably, additional in the IR/NIR range 34.

For noise reduction and reproducible measurement conditions (operating point), the light source 5 can be deactivated over a period of, e.g., at least 1 second, 10 seconds, or a minute, before activation in order to enable cooling down of the spectrometer 6.

The light from the light source 5 guided to the chamber 26 is at least partially reflected, scattered, or a Raman scattered or a fluorescence is exited causing light originating from the chamber 26 to the spectrometer 6 being measured as spectrum 40.

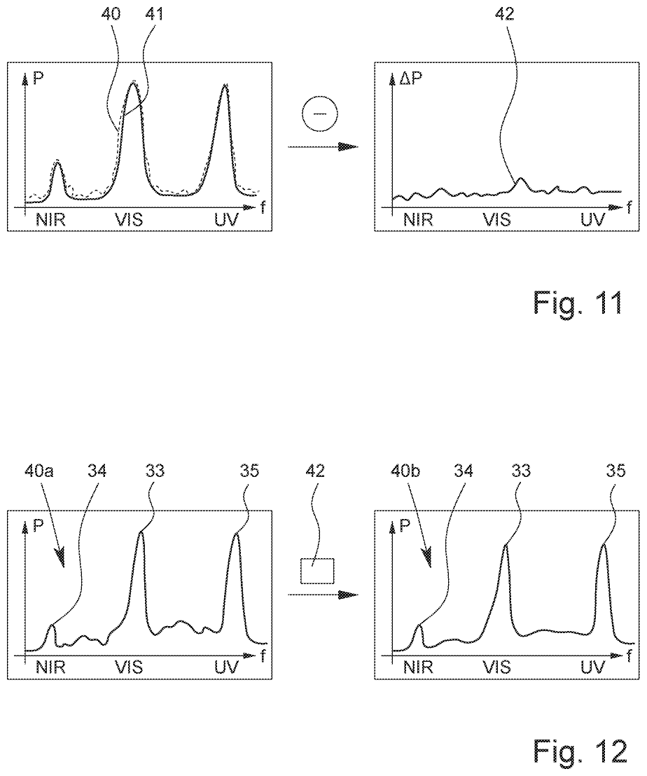

Referring to FIG. 11, for calibration the measured spectrum 40 (preferably with empty chamber) can be compared with a (calibration) reference spectrum 41 and, on the basis of a difference between the measured spectrum 40 and the (calibration) reference spectrum 41, a correction variable 42 like the conversion matrix can be determined for correcting spectra 40 measured with an element E of body fluid or tissue or environmental sample comprised in the chamber 26.

The correction preferably converts the measured spectrum 40 to a corrected spectrum 40 with characteristics as it was measured with a reference spectrometer by means of which reference probes are or were characterized, forming reference spectrum 41A-parameter P-pairs used for finding a parameter P by correlation of the corrected measured spectrum 40 with the reference spectrum 41A having assigned the corresponding parameter P searched for.

Filtering (digital or optical) of particular spectral bands of light optionally can be provided and/or used in order to maximize the correlation.

There are two kinds of reference spectra 41, 41A used in the present invention. One reference spectrum 41 is used in the calibration process to find the correction variable 42 by comparison of the spectrum 40 measured preferably with empty chamber 26 with an expected reference spectrum 41, e.g. of a reference spectrometer. The other reference spectrum 41A is the one measured with a reference spectrometer having a corresponding parameter P assigned thereto enabling finding a specific parameter P by correlation of a measured spectrum 40 with said reference spectrum 41A. Thus, different reference spectra 41, 41A are used during calibration on the one hand and during parameter P finding on the other hand.

Before or while the spectrum 40 is measured, the brightness of the light source 5 can be reduced and/or feedback controlled.

Referring to FIG. 7, in the embodiment shown, the maximum in the VIS range 33 is clipped. That is, the maximum has power overshooting the dynamic range of the spectrometer 6, causing an output spectrum 40 of the spectrometer 6 having a saturation range at a maximum output value. If such clipping behavior is detected during a check for clipping, the brightness control 36 reduces the brightness, preferably only of the LED 5A, 5B which is responsible for the clipping, in the example shown LED 5A causing the clipping at the maximum in the VIS range 33 (cf. FIG. 9 again).

The clipping check and the brightness reduction can form the feedback control loop 38 enabling reduction of the peak power in such an extent that the reduced brightness is such that the spectrometer 6 is driven at least substantially free of overdrive and/or up to the limit of the spectrometer's 6 dynamic range already discussed based on FIG. 5. This can be done with either or both of the LEDs 5A, 5B. That is, there can be separate feedback loops 37, 38 for feedback controlling the LEDs 5A, 5B such that the maximum in the VIS range 33 and the maximum in the UV range 35 both are reduced and/or controlled such that the spectrometer 6 is driven at least substantially free of overdrive and/or up to the limit of the spectrometer's 6 dynamic range at at least one of the maximum in the VIS range 33 and the maximum in the UV range 35, particularly both.

FIG. 8 shows the result of such feedback control where the maximum in the VIS range 33 emitted by one LED 5A and the maximum in the UV range 35 emitted by a different LED 5B each are controlled such that the resulting spectrum 40 measured by the spectrometer 6 essentially makes use of the complete dynamic range of the spectrometer 6.

In conclusion, referring to FIG. 9 again, it is preferred that during calibration the brightness of the light source 5, in particular the brightness of at least one or both of the LEDs 5A, 5B, is (a) a feedback controlled brightness or (b) is a brightness reduced compared to its nominal brightness, and/or (c) is a brightness reduced compared to the brightness while a spectrum 40 of the light originating from the element E and/or chamber 26 is measured with the spectrometer 6.

Further preferably, both LEDs 5A, 5B for generating light of different wavelength ranges UV, VIS, NIR, are operated with a reduced brightness or a brightness being reduced compared to the LED's nominal brightness and/or brightness of the LEDs 5A, 5B when measuring an element E while a spectrum 40 of the light originating from the element E and/or chamber 26 is measured with the spectrometer 6.

Generally speaking, it is preferred that the brightness is a reduced brightness or is reduced to such an extent that the spectrometer 6 is driven at least substantially without overmodulation (clipping) and/or up to the limit of its dynamic range.

Optionally, in the calibration process as shown in FIG. 9 the spectrum 40 can be checked for plausibility and/or a correction variable like a conversion matrix can be derived based on the spectrum 40 for correction of future spectra measurements.

The calibration of the spectrometer 6 can be performed while a parameter P of the element E can be determined subsequently with the spectrometer 6.

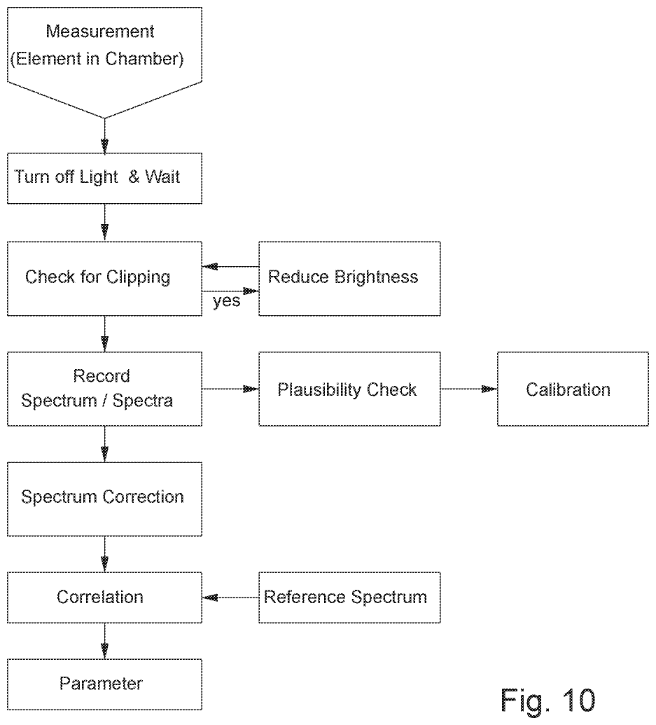

The element E preferably is measured based on the procedure depicted in FIG. 10.

Optionally, the light source 5 can be turned out for a period of time like a few seconds to avoid thermal issues like drift of operating points and noise before measuring the spectrum 40.

By illuminating an element E received in the chamber 26 with the light source 5, the spectrum 40 of light originating from the element E is measured with the spectrometer 6.

The measured spectrum 40 can be converted to a corrected spectrum 40 by correcting the measured spectrum 40 with the correction variable 42. The correction variable 42 preferably has been determined by means of the calibration, in particular as conversion matrix.

The corrected spectrum 40 can be correlated with one or more (parameter) reference spectra in order to determine a parameter P representing property of the element E.

In a further aspect of the present invention, the spectrometer 6 of the optical system S is monitored with the regard to change in its measurement behavior, and, upon detection of the change in the measurement behavior of the spectrometer 6, a calibration, preferably as discussed with regard to FIG. 9, is requested or performed. In particular, the spectra 40 of preceding measurements are compared with each other for monitoring the function of the spectrometer 6 in order to detect the change in the measurement behavior.

Parameters P representing a property of different reference elements E can be determined in advance with the reference method, and (parameter) reference spectra 41A of the respective elements E preferably are measured with a reference spectrometer. The parameter P of the element E held in the chamber 26 is then determined by correlating the corrected spectrum 40 with the reference spectra 41A. This can be achieved or improved with self-learning methods, in particular using machine learning, neural networks, and/or artificial intelligence.

The monitoring can be realized with a plausibility check of the recorded spectrum 40 as depicted in FIG. 10. Alternatively, or additionally, a proper functioning of the spectrometer 6 is monitored by comparison of the spectrum 40 with spectra 40 which have been previously determined using the spectrometer 6. The plausibility check can be realized or based on such comparison.

If the plausibility check fails, i.e., if the recorded spectrum 40 is not comply with particular expectations or varies in an unexpected manner from earlier recorded spectra 40, the recalibration, i.e. starting the calibration again can be initiated.

By recalibration, a (new) correction variable 42 (conversion matrix or different correction measure) can be derived from the recorded spectrum 40, in particular, by comparison with one or more reference spectra 41. This reference spectrum 41 for calibration preferably complies with a spectrum expected from a reference spectrometer under the same conditions. This enables correction of spectra 40 measured from elements E afterwards, in particular to compensate for parasitic effects a real sensor measuring the spectrum 40 might have.

A quality indicator 43 can be determined and/or output like depicted in FIG. 5. The quality indicator 43 preferably is output with the measured spectrum 40 or with the parameter P determined using this spectrum 40.

The quality indicator can be assigned to the measurement/measured spectrum 40, to the parameter P determined based on the spectrum 40, or to a further result deducted from spectrum 40.

The quality indicator 43 can be determined by means of the plausibility examination of the spectrum 40 and/or the comparison with the spectra 40 which have been previously determined using the spectrometer 6 by which the plausibility check can be realized or which the plausibility check can comprise.

When measuring the spectrum 40, for calibration and/or for measurement, it is preferred to perform one or more noise reduction measures. Reducing noise in the spectrum 40 results in a better signal to noise ratio and, thus, to a more reliable and exact spectrum 40 enabling a more reliable and exact parameter P determination based on the measured spectrum 40.

It is preferred to reduce the power loss of the spectrometer 6 by temporary deactivation, preferably directly before starting a measurement. This helps reduce sensor temperature and, thus, thermal noise.

Alternatively, or additionally spectra 40 from several measurements with the spectrometer 6 of the same element E are combined, in particular averaged. This enables compensation of random effects.

Alternatively, or additionally the optical system 1 is calibrated, preferably such that the signal-to-noise ratio is increased.

In particular, alternatively or additionally, the optical system 1 is calibrated to driving the spectrometer 6 at least substantially overload-free up to the limit of its dynamic range so that the signal-to-noise ratio is optimized.

Alternatively, or additionally, a temperature of the spectrometer 6, a temperature increase and/or a temperature drift of the spectrometer 6 is reduced or limited. This can be achieved by establishing a waiting time with preferably deactivated light source 5 before the calibration is started, before the measurement is started, and/or between the calibration and the measurement are started.

The optical system 1 comprising a chamber 26 for receiving the element E of body fluid or tissue or environmental sample to be characterized by the optical system 1, the light source 5 for illuminating the chamber 26 with light and the spectrometer 6 for measuring a spectrum 40 of light originating from the chamber 26 preferably is adapted to perform a method according to the above aspects regarding calibration and/or noise reduction. This aspect can be combined with further aspects discussed before and hereafter.

In particular it is preferred that the optical system 1 comprises the light source 5 comprising at least two LEDs 5A, 5B of different light color/wavelength ranges/spectral ranges UV, VIS, NIR and at least three fiber optics 28, wherein light from the two LEDs 5A, 5B is guided to the chamber 26 via two of the three fiber optics 28 and light from the chamber 26 is guided to the spectrometer 6 via a third of the three fiber optics 28. Using at least two different wavelength (range) maxima 33, 34, 35 excited by at least two LEDs 5A, 5B has turned out as particular advantageous to enable measuring a spectrum 40 with high signal to noise ratio/resolution which supports the calibration and/or noise reduction.

In order to determine the parameter P representing a property of the element E, in on light having at least two spectral maxima 33, 34, 35 of different wavelength ranges UV, VIS, NIR is directed onto the element E, a spectrum comprising reflected components of the light, scattered components of the light, and/or light caused by Raman scattering and/or fluorescence of the element E is measured with the spectrometer 6, and the parameter P is determined by evaluating the spectrum 40.

Particular elements E have been turned out to be difficult, imprecise to be determined by means of systems known in the art. The system 1 and methods according to the present invention, however, are capable of such measurement and parameter P determination in a synergistic manner. Using the system 1 and methods according to the present invention have been turned out to be particularly advantageous where the element E:
a. is or comprises bird blood, preferably EDTA- and/or heparin-anticoagulated bird blood, wherein the parameter is determined characterizing one or more properties concerning:
   Hematocrit
   Hemoglobin
   erythrocytes
   erythrocyte indices (MCH, MCHC, MCV)
   Platelets
   Leukocytes incl. differentiation (heterophilic, basophilic and eosinophilic granulocytes, lymphocytes, monocytes)
b. is or comprises serum, meat juice or saliva of a pig, wherein the parameter is determined characterizing one or more properties concerning:
   Androstenone
   Skatol
c. is or comprises oral fluid, saliva or meat juice of a pig, wherein the parameter is determined characterizing one or more properties concerning:
   Cortisol
   Haptoglobin
   C-reactive protein
d. is or comprises saliva, faeces or serum of an animal, wherein the parameter is determined characterizing one or more properties concerning:
   Progesterone
   17-OH-progesterone
   estradiol.

Different aspects of the present invention can be combined and such combinations can result in synergistic advantageous effects even if of such combinations and/or effects discussion are not mentioned explicitly.

List of reference signs:

| | |
|---|---|
| 1 | computer |
| 2 | hub |
| 3 | usb |
| 4 | LCD display and control; |
| 5 | Light source |
| 6 | spectrometer |
| 7 | plug-in/plug-out system |
| 8 | Optical bench |
| 9 | Lithium-ion battery; and |
| 10 | attachment for modular probes |
| 11 | hole |
| 12 | window |
| 13 | glass |
| 14 | attaching section |
| 15 | attaching section |
| 16 | window |
| 17 | pressure plug |
| 18a | mirror |
| 18b | mirror |
| 18c | mirror |
| 19 | axis of the attachment |
| 20 | direction |
| 21 | optical bench |
| 22 | capsule |
| 23 | micro-channel |
| 24 | puncturing tip |
| 25 | mirror(s) |
| 26 | chamber |
| 27 | fast plug-in/plug out system |
| 28 | fiber optics |
| 29 | illuminating and center capture fiber optics |
| 30 | focusing lenses |
| 31 | fast plug- in/plug out system |
| 32 | fast plug-in/out system |
| 33 | first maximum |
| 34 | second maximum |
| 35 | third maximum |
| 36 | brightness control |
| 37 | control loop |
| 38 | control loop |
| 39 | reference variable |
| 40 | spectrum |
| 40A | corrected spectrum |
| 41 | reference spectrum (calibration) |
| 41A | reference spectrum (correlation) |
| 42 | correction variable |
| 43 | quality indicator |
| E | element |
| S | system |
| P | parameter |

What is claimed is:

1-15. (canceled)

16. An optical system comprising:
a chamber for receiving an element to be characterized by the optical system,
a light source for illuminating the chamber, and
a spectrometer for recording a spectrum of light issuing from the chamber,
wherein the light source comprises at least two separate LEDs that emit light having at least two spectral maxima in different wavelength ranges, the light source being coupled to the chamber such a manner that light is guided from the light source to the chamber when the light source is activated.

17. The optical system according to claim 16, further comprising at least three-fiber optics, wherein light with the spectral maxima in at least two different wavelength ranges is guided to the chamber via a first and second of the three-fiber optics and light is guided from the chamber to the spectrometer via a third of the three-fiber optics.

18. The optical system according to claim 16, wherein the light source is configured to generate light with spectral maxima in a wavelength in the UV range and in a wavelength range visible to the human eye.

19. The optical system according to claim 16, wherein the light source is configured to generate light with spectral maxima in a wavelength in the UV range and in an infrared range.

20. The optical system according to claim 19, wherein the light source has a UV LED which is configured for generating light with a spectral maximum in the UV wavelength range in addition to generating light with a spectral maximum in the infrared wavelength range (IR).

21. The optical system according to claim 20, wherein the UV LED is coupled to the chamber via fiber optics, via which the light generated or producible by the UV LED with the maxima in the UV range and in the infrared range is guided to the chamber while being superimposed.

22. The optical system according to claim 16, wherein the spectrometer comprises a brightness control for controlling a brightness of the light source.

23. The optical system according to claim 22, wherein the brightness control is designed to control the brightness in such a way that the spectrometer is controlled at least substantially free of overdrive and/or up to the limit of its dynamic range at at least one of the maxima in the different wavelength ranges.

24. The optical system according to claim 22, wherein the brightness control is designed to individually control the brightness of the maxima in at least two of the different wavelength ranges in such a way that the spectrometer at at least one of the maxima of the different wavelength ranges is driven at least substantially free of overdrive and/or up to the limit of its dynamic range.

25. The optical system according to claim 22, wherein the brightness control forms at least one control loop, wherein the brightness control is coupled to the spectrometer for measuring brightness of the light received by the spectrometer, and wherein the brightness control is coupled to the light source for controlling the brightness of the light source in such a way that brightness of the light source is controllable with the brightness control on the basis of a comparison of the brightness measured with the spectrometer with a reference variable representing the dynamic range of the spectrometer.

26. The optical system according to claim 22, wherein the brightness of the light with a wavelength in the UV range (UV) and of the light with a wavelength in the wavelength range visible to the human eye (VIS) are separately controllable by feedback control loops.

27. A method for determining a parameter representing a property of the element with an optical system, comprising a chamber for receiving an element to be characterized by the optical system, a light source for illuminating the chamber with light, and a spectrometer for measuring a spectrum of light originating from the chamber,
the method comprising, in order to determine the parameter representing a property of the element, generating light having at least two spectral maxima in different wavelength ranges by separate LEDs, directing the light onto the element, measuring a spectrum of reflected components of the light, scattered components of the light, and/or light caused by Raman scattering or fluorescence of the element with the spectrometer, and determining the parameter by evaluating the spectrum.

28. The method according to claim 27, wherein the at least two spectral maxima in different wavelength ranges are, on the one hand, in a UV wavelength range, and on the other hand, in a wavelength range visible to the human eye (VIS).

29. The method according to claim 27, further comprising controlling the brightness of the light source by a feedback control or with a brightness that is reduced compared to a nominal brightness of the light source and/or a brightness that is reduced compared to a brightness of the light source while measuring the spectrum of an element with the spectrometer.

30. The method according to claim 27, wherein the element
a. is or comprises bird blood, preferably EDTA- and/or heparin-anticoagulated bird blood, wherein the parameter is determined characterizing one or more properties concerning:
Hematocrit
Hemoglobin
erythrocytes
erythrocyte indices (MCH, MCHC, MCV)
Platelets
Leukocytes incl. differentiation (heterophilic, basophilic and eosinophilic granulocytes, lymphocytes, monocytes)
b. and/or is or comprises serum, meat juice or saliva of a pig, wherein the parameter is determined characterizing one or more properties concerning:
Androstenone
Skatol
c. and/or is or comprises oral fluid, saliva or meat juice of a pig, wherein the parameter is determined characterizing one or more properties concerning:
Cortisol
Haptoglobin
C-reactive protein
d. and/or is or comprises saliva, faeces or serum of an animal, wherein the parameter is determined characterizing one or more properties concerning:
Progesterone
17-OH-progesterone
estradiol.
